(19) United States
(12) Patent Application Publication
(10) Pub. No.: US 2009/0088845 A1
(43) Pub. Date: Apr. 2, 2009

(54) TITANIUM TANTALUM OXYGEN ALLOYS FOR IMPLANTABLE MEDICAL DEVICES

(76) Inventors: Stanley Abkowitz, Lexington, MA (US); Susan M. Abkowitz, Burlington, MA (US); Harvey Fisher, Lexington, MA (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/237,164

(22) Filed: Sep. 24, 2008

Related U.S. Application Data
(60) Provisional application No. 60/960,295, filed on Sep. 24, 2007.

(51) Int. Cl. A61F 2/02; C01G 23/04

(52) U.S. Cl. 623/11.11; 423/598

(57) ABSTRACT

The present disclosure relates to alloys of titanium, tantalum, and oxygen that include from greater than 0 to about 60 weight % tantalum, from greater than or equal to about 0.10 weight % oxygen, and the balance titanium and other impurities. Also disclosed are methods of making such alloys. In addition, articles of manufacture including such alloys, such as biomedical implants, are disclosed.

This application claims benefit of priority to U.S. Provisional Application No. 60/960,295, filed Sep. 24, 2007, the contents of which are incorporated herein by reference.

The present disclosure generally relates to alloys of titanium, tantalum, and oxygen. In addition, the present disclosure relates to methods of manufacturing alloys of titanium, tantalum, and oxygen, as well as biomedical implants and other articles of manufacture including such alloys.

The most common materials used for medical implants include metallic alloys, ceramics and composites formed of biocompatible polymers and various reinforcing materials. Ideally, a biomedical implant material should exhibit a combination excellent biocompatibility, corrosion resistance, strength, ductility, and an elastic modulus that approximates that of human bone. As described below, however, prior developed materials generally do not exhibit a satisfactory combination of these properties for load bearing applications. In particular, these prior known materials generally do not exhibit a desirable combination of high strength, relatively low elastic modulus, good ductility and in some cases, shape memory or superelasticity.

For example, polymeric materials have been developed for use in biomedical implants such as intraocular lenses, facial bone remodeling and other non-load bearing applications. Although these materials exhibit low elastic modulus relative to metallic and ceramic materials, their intrinsic strength is too low for load-bearing implant applications. While the strength of these materials can be enhanced by the inclusion of reinforcements such as filler materials, such reinforcements often degrade the other mechanical properties of the material.

Ceramics and metallic alloys (e.g., stainless steel, cobalt alloys, and the Ti-6Al-4V alloy) have also been developed for use in biomedical implants. These materials have been proven to provide excellent corrosion resistance and high compression strength for load-bearing implant applications. However, while these materials have the requisite strength to perform well in load bearing applications, they typically exhibit less than optimum biocompatibility, elastic modulus and/or ductility.

For example, common alloy materials such as 316 stainless steel (elastic modulus ˜200 GPa), cast heat-treated Co—Cr—Mo alloy (elastic modulus ˜240 GPa), and the Ti-6Al-4V alloy (elastic modulus ˜111 GPa) exhibit an elastic modulus that is substantially greater than that of human bone. As a result, biomedical implants manufactured from these materials do not effectively transfer stresses imparted during normal use to surrounding bone tissue. This “bone shielding” can result in decalcification, localized thinning (resorption), and weakening of the bone structure surrounding an implant made of these materials.

In addition, many known metal alloys currently used for biomedical implants contain elements such as aluminum, vanadium, cobalt, nickel, molybdenum, and chromium. These elements have the potential to leech out of an installed implant and into the surrounding tissues and/or vascular system of a patient. Thus, implants made of these materials may cause long term deleterious effects to a patient. As a result, their biocompatibility is suspect.

To address the issues with common metallic implant materials, research has considered metallic titanium and tantalum for biomedical applications. Although these materials have excellent biocompatibility, their strength is relatively low compared to commonly used biomedical alloy materials. Thus, these materials are of limited usefulness in load bearing applications, such as biomedical implants.

Binary titanium tantalum (TiTa) alloys have also been investigated for biomedical applications in view of their desirable properties such as excellent biocompatibility, corrosion resistance, low modulus, and potential for shape memory behavior and superelasticity. Research has shown that as tantalum is added to titanium metal in greater proportion, the Young's Modulus of the resultant titanium tantalum composition changes. This change is demonstrated in FIG. 1, which shows that the dynamic Young's Modulus of binary TiTa reaches a minimum when the alloy contains 30 weight % of tantalum. This low modulus makes Ti-30Ta alloy potentially useful for medical implants that benefit from low modulus. As shown in Table 1 below, however, binary TiTa alloys exhibit low strength relative to other titanium alloys.

TABLE 1
Properties of Commonly Used Medical Implant Alloys

| | Ultimate | | |
|---|---|---|---|
| | Tensile | | |
| | Strength | Elastic modulus | Strength-to- |
| Alloy | (MPa) | (GPa) | Modulus Ratio |
| Commercially pure Ti | 345 | 103 | 3.35 |
| F75 (CoCrMo) | 655 | 220 | 2.98-3.14 |
| 316 stainless steel | 580-1034 | 193 | 3.00-5.36 |
| Ti—6Al—4V | 965 | 114 | 8.77 |
| Ti—6Al—7Nb | 960 | 111 | 8.65 |
| Ti—I3Nb—13Zr | 862 | 79 | 10.91 |
| Ti—30Ta | 587 | 69 | 8.51 |

Because of their relatively low strength, the usefulness of binary TiTa alloys is limited with respect to load bearing applications. In addition, these alloys are very difficult to prepare by conventional methods because of the disparity in melting points and density between titanium (density=4.5 g/cm³; melting point=1670° C.) and tantalum (density=16.6 g/cm³; melting point=3020° C.).

Thus, there is a need in the art for improved materials that exhibit desirable properties for biomedical applications, such as biocompatibility, corrosion resistance, strength, ductility, elastic modulus, and in some cases, shape memory or superelasticity. In particular, there is a need for materials that exhibit an improved combination of high strength, good ductility, relatively low elastic modulus, and good ductility.

### SUMMARY OF THE INVENTION

The present disclosure provides alloys of titanium, tantalum, and oxygen (TiTaO) that are suitable for use as a biomedical material for implantable medical devices, as well as in other articles of manufacture. Applications for these alloys include those in which the combination of high strength, ductility, and relatively low elastic modulus is desired. For example, the alloys of the present disclosure may be suitable for use in orthopedic, dental, and vascular implants, as well as in aerospace, automotive, nuclear, power generation, jewelry, communication, and chemical processing applications.

Thus, consistent with the present disclosure are TiTaO alloys that include from greater than or equal to about 20 to about 60 weight % tantalum, greater than or equal to about 0.1 weight % oxygen, and the balance titanium and incidental impurities. For example, some embodiments of the TiTaO alloys disclosed herein include from about 20 to about 40 weight % tantalum, from about 0.10 to about 0.30 weight % oxygen, and from about 60 to about 80 weight % titanium and incidental impurities. In additional embodiments, the TiTaO alloys disclosed herein include from about 40 to about 60% tantalum, from about 0.1 to about 0.3 weight % oxygen, and from about 40 to about 60 weight % titanium and incidental impurities.

The alloys disclosed herein can exhibit a desirable properties for biomedical implant and other applications. For example, some embodiments of the disclosed alloys exhibit at least one of excellent biocompatibility, high strength, good ductility, and relatively low elastic modulus. Further, in some embodiments the TiTaO alloys disclosed herein exhibit austenite to martensite transitions that are indicative of shape memory and/or superelastic properties.

Also consistent with the present disclosure are methods for making TiTaO alloys and articles of manufacture including such alloys. For example, these alloys may be made by powder metallurgy followed by subsequent hot working, such as extrusion, rolling, and/or forging. In some embodiments, the alloys disclosed herein are subjected to a heat treatment process.

It is to be understood that both the foregoing general description and the following detailed description are exemplary and explanatory only and are not restrictive of the invention, as claimed.

### BRIEF DESCRIPTION OF THE DRAWINGS

FIG. 1 is a plot of dynamic Young's Modulus vs. tantalum content for binary TiTa alloys known in the art.

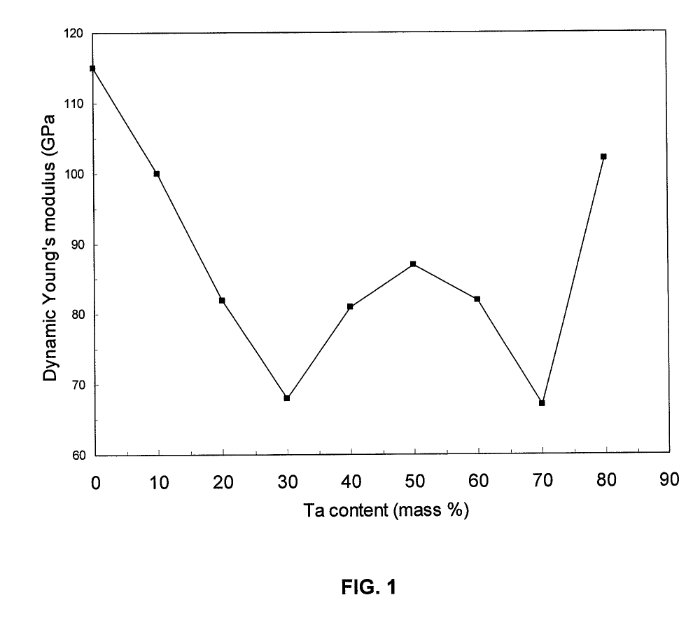

FIG. 2 is a phase diagram for binary titanium tantalum.

FIG. 3. plots 2Θ vs. Intensity for several TiTaO alloys according to the present disclosure.

FIG. 4 plots Yield Strength vs. Tantalum Composition for TiTaO alloys according to the present disclosure and comparable binary TiTa alloys.

FIG. 5. plots Young's Modulus vs. Tantalum Composition for TiTaO alloys according to the present disclosure and comparable binary TiTa alloys.

### DESCRIPTION OF THE EMBODIMENTS

Reference will now be made in detail to exemplary embodiments of the invention.

One aspect of the present disclosure relates to TiTaO alloys that have elevated oxygen content. As used herein, the phrase “elevated oxygen content” means that an alloy contains greater than or equal to about 0.1 weight % of oxygen.

Thus, consistent with the present disclosure are TiTaO alloys that include from greater than 0 to about 60 weight % tantalum, greater than or equal to about 0.1 weight % oxygen, and the balance titanium and incidental impurities. For example, the alloys disclosed herein may include from about 20 to about 40 weight % tantalum, from about 0.10 to about 0.30 weight % oxygen, and from about 60 to about 80 weight % titanium and incidental impurities. Further, the TiTaO alloys described herein disclosure may include from about 40 to about 60 weight % tantalum, from about 0.1 to about 0.3 weight % oxygen, and from about 40 to about 60 weight % titanium and incidental impurities.

The tantalum and titanium content of the alloys according to the present disclosure may vary to any degree within above described ranges, such as continuously or in 1, 3, and 5 weight % increments. In some embodiments, tantalum is present in an amount ranging from 25-35% by weight, such as from 28 to 32% by weight. In additional non-limiting embodiments, tantalum and titanium are present in amounts ranging from about 35 to about 60% by weight, such as about 35 to about 55% by weight, or even about 45 to about 55% by weight. In further non-limiting embodiments, titanium and tantalum are present in the TiTaO alloys described herein in equal or near equal amounts.

The oxygen content of the alloys according to the present disclosure may also vary to any degree within the above described ranges, such as continuously or in 0.01 weight % increments. In some embodiments, the TiTaO alloys of the present disclosure may contain oxygen in an amount ranging from about 0.1 to about 0.3 weight %, such as from greater than or equal to 0.1 weight %. For example, some non-limiting embodiments of the TiTaO alloys according to the present disclosure contain about 0.2 weight % oxygen.

The TiTaO alloys described herein may exhibit excellent mechanical properties for biomedical and other applications. For example, the alloys described herein may exhibit at least one of excellent biocompatibility, Yield Strength, Ultimate Tensile Strength, ductility, Young's Modulus, Shear Modulus, and strength to modulus ratio for use in biomedical and other applications. In some embodiments, the alloys described herein exhibit a desired combination of high Yield Strength, low Young's Modulus, and good ductility.

For example, the alloys of the present disclosure may exhibit a Yield Strength (YS) that is suitable for biomedical and other applications. For example, the alloys according to the present disclosure may have a YS ranging from greater than or equal to about: 600 MPa; 650 MPa; 700 MPa; 750 MPa; 800 MPa; and 850 MPa. In some embodiments, the TiTaO alloys of the present disclosure exhibit a Yield Strength of greater than or equal to 854 MPa.

The alloys according to the present disclosure may also exhibit an Ultimate Tensile Strength (UTS) that is suitable for biomedical and other applications. For example, the UTS of the TiTaO alloys described herein may, for example, range from greater than or equal to about: 800 MPa; 850 MPa; 900 MPa; 950 MPa; 1000 MPa; and 1050 MPa. In some embodiments, the TiTaO alloys of the present disclosure have an UTS greater than or equal to about 1069 MPa.

The TiTaO alloys according to the present disclosure may also exhibit good ductility/elongation. For example, the ductility of the TiTaO alloys described herein may, for example, range from greater than or equal to 6% to about 20%, such as from about 7% to about 13%, including from about 8% to about 12%. In some embodiments, the TiTaO alloys according to the present disclosure may exhibit greater than 17% elongation, or even greater than 20% elongation.

The Young's Modulus (YM) of the TiTaO alloys according to the present disclosure may also be suitable for biomedical and other applications. For example, the alloys described herein may have a YM ranging from less than or equal to about 100 GPa, such as less than or equal to about 75 GPa, or even less than or equal to about 50 GPa. In some embodiments the TiTaO alloys according to the present disclosure exhibit a YM ranging from about 45 to about 100 GPa, such as about 45 to about 76 GPa, including about 45 to about 69 GPa, for example about 45 to about 69 GPa, or even about 45 to about 51 GPa.

The TiTaO alloys according to the present disclosure may also exhibit a Shear Modulus (SM) that is suitable for biomedical and other applications. For example, the SM of the TiTaO alloys described herein may range from about 28 to about 36 GPa, such as about 20 to about 35 GPa. In some embodiments, the Shear Modulus of the TiTaO alloys according to the present disclosure is about 31 GPa.

The TiTaO alloys according to the present disclosure may also exhibit a higher strength-to-modulus ratio, than that of binary TiTa. For example, the TiTaO alloys according to the present disclosure may exhibit a strength-to-modulus ratio ranging from about: 9.0 to about 17.5, such as from about 9.5 to about 13, or even from about 10.5 to about 12.0. In some embodiments, the TiTaO alloys according to the present disclosure exhibit a strength-to-modulus ratio greater than or equal to about 17.3. In contrast, binary TiTa exhibits a strength to modulus ratio of 8.5

In addition, the TiTaO alloys disclosed herein may exhibit improved strength relative to binary TiTa alloys, while preserving the other desirable properties of binary TiTa, such as biocompatibility, ductility, low modulus, and in some instances, shape memory properties such as superelasticity. In other words, the alloys of the present disclosure may exhibit a significantly improved combination of properties for biomedical and other applications, such as high strength, relatively low elastic modulus, high ductility, and in some instances, shape memory and superelasticity. The ability of the alloys disclosed herein to exhibit, for example, an improved combination of high strength, low modulus, and improved ductility is contrary to the general belief in the field that low oxygen levels (e.g., less than 0.1 weight %) are necessary in titanium alloys in order to achieve an optimal combination of strength, elastic modulus, and ductility.

Also, consistent with the present disclosure are TiTaO alloys that exhibit austenite to martensite transformations that are indicative of shape memory properties and superelasticity. In some non-limiting embodiments, the TiTaO alloys of the present disclosure that have these austenite to martensite transformations include about 35 to about 55 weight % tantalum and from greater than or equal to about 0.1 weight % oxygen. For example, some embodiments of these alloys contain from about 45 to about 55 weight % tantalum, from about 0.1 to about 0.3 weight percent oxygen, and the balance titanium and incidental impurities. These alloys are capable of forming medical devices that benefit from such shape memory properties, including nickel free stent components that have significantly improved strength relative to binary TiTa alloys, such as binary Ti-30Ta. The nickel-free nature of some embodiments of these alloys is beneficial, because a portion of the human population is hypersensitive (i.e., allergic) to nickel.

Another aspect of the present disclosure are directed to articles of manufacture that include the TiTaO alloys described herein. For example, the TiTaO alloys of the present disclosure may be used to form all or a portion of parts, tools, equipment, and other articles suitable for biomedical applications, aerospace applications, automotive applications, nuclear applications, power generation applications, jewelry, communications, and chemical processing applications.

In some embodiments, the TiTaO alloys of the present disclosure may be used to form all or a part of components, parts, tools, instruments, and equipment for use in orthopedic, dental and vascular applications, such as partial and/or total joint replacement procedures, fracture fixation, cardiovascular procedures, restorative and reconstructive dental procedures, spinal fusion and/or spinal disc replacement procedures. For example, the TiTaO alloys described herein may be used to manufacture all or a part of a stent such as a tubular body or scaffolding, an intermedullary rod, a fracture plate, a spinal fixation replacement component, a spinal disc replacement component, a fastener such as a screw or a nail, a plate, a wire, a cable, an anchor (e.g., an orthodontic anchor), a dental casting, a dental implant, an orthodontic arch wire, a heart valve component, such as a ring, and profile and plate stocks. In some embodiments, the TiTaO alloys described herein exhibit shape memory properties such as superelasticity and/or austenite to martensite transformation, and form all or a portion of a stent, such as a tubular body or scaffolding.

Also consistent with the present disclosure are parts, tools, instruments, and equipment for non-biomedical applications, and which are totally or partially formed from the TiTaO alloys of the present disclosure. For example, the alloys described herein may be used to form all or a portion of automotive and motorcycle springs, automotive torsions bars, aerospace fasteners, corrosion-resistant thin sheet for military and commercial aircraft, and corrosion-resistant chemical processing tubing and fasteners.

It is to be appreciated that the alloys described herein may be used in any other application for which titanium alloys are typically used.

Another aspect of the present disclosure relates to methods of producing TiTaO alloys that include increased amounts of oxygen. For example, the alloys described herein may be manufactured by powder metallurgy followed by optional hot-working processes such as rolling, forging, and/or extrusion.

In some non-limiting embodiments of the present disclosure, the TiTaO alloys described herein are manufactured by a powder metallurgy process. In this process, the alloys described herein are manufactured by blending a titanium containing powder with a tantalum containing powder to form a blended powder; compacting the blended powder; consolidating the blended and compacted powder by at least one process chosen from pressing, sintering and hot isostatic pressing; optionally hot working the consolidated material by at least one process selected from forging, rolling, and extruding; and optionally heat treating the resultant article.

The TiTaO alloys disclosed herein may be subject to an optional heat treatment process to improve the homogeneity of the alloy. For example, the alloys of the present disclosure may be heat treated in a vacuum for 24 hours at about 1200° C. In some embodiments, the heat treatment also assists in the development of the tensile properties of the material. In some embodiments, the alloys described herein show an increase in tensile strength after heat treatment, which may be due to solution hardening.

Other than in the examples, or where otherwise indicated, all numbers expressing endpoints of ranges, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the present disclosure are approximations, unless otherwise indicated the numerical values set forth in the specific examples are reported as precisely as possible. Any numerical value, however, inherently contains certain errors necessarily resulting from the standard deviation found in their respective testing measurements.

The disclosure will be more fully illustrated using the following non-limiting examples.

### EXAMPLES

TiTaO alloys comprising tantalum in an amount ranging from 20 to 60% by weight, about 0.2% by weight of oxygen, and the balance titanium, were manufactured to full density using a powder metallurgy process. Specifically, titanium powder containing a known amount of oxygen was blended with varying amounts of tantalum powder. The blended powder was compacted to form a compacted body, which was then sintered and hot isostatically pressed. The resulting samples were subjected to heat treatment in a vacuum at 1200° C. for 24 hours to improve their homogeneity. The metallurgical and mechanical properties of the alloys were analyzed. The results of these analyses are set forth below.

### Metallurgical Analysis of the Produced Samples

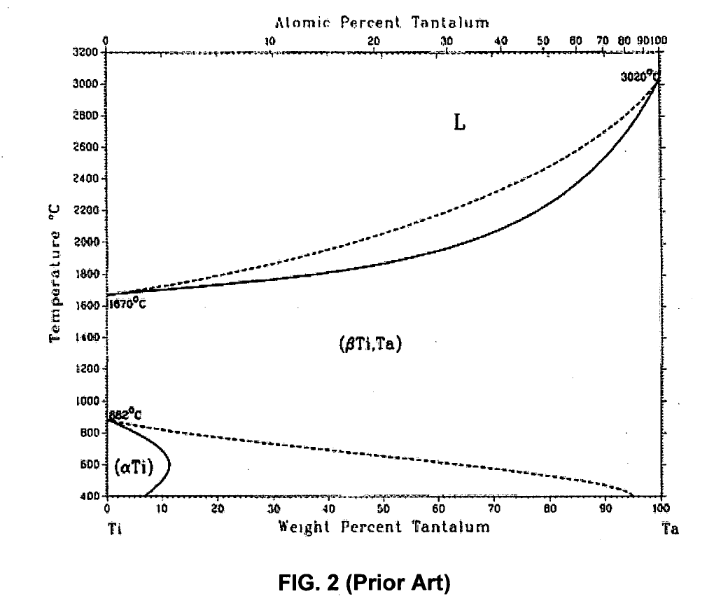

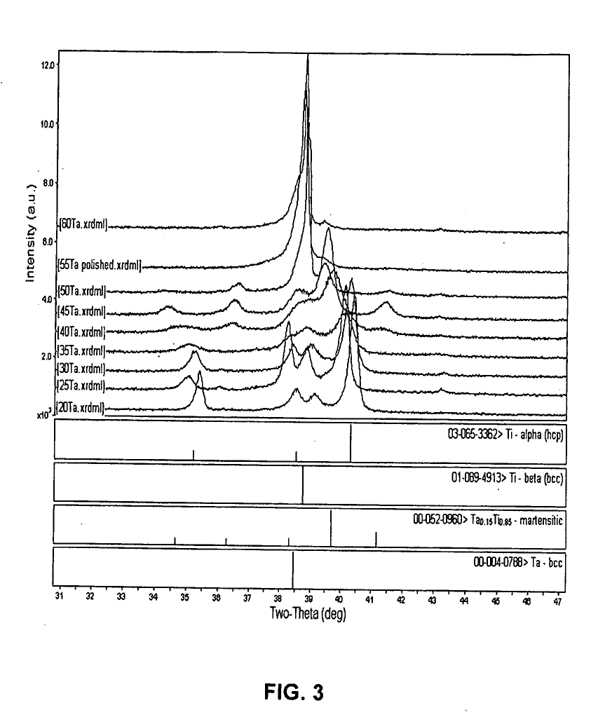

The crystal structures of the manufactured alloy samples were predicted using the phase diagram for binary TiTa shown in FIG. 2. The alloys were then subject to x-ray diffraction (XRD) analysis to determine the actual phases present in the material. The measured XRD spectra (intensity vs. 20) for these alloys is reproduced in FIG. 3. The peaks were analyzed using diffractometer software and identified with known phases from Ti and Ta. The predicted phase(s) vs. the measured phase(s) are shown in Table 2 below.

TABLE 2
Measured vs. Predicted Phases in TiTaO
Alloys Containing 0.2 wt % Oxygen

| Tantalum | | |
|---|---|---|
| Composition | Phases predicted | |
| range (weight %) | from FIG. 2 | Measured Phases |
| 20-35% | Mix of α and β Ti | Mix of α and β Ti |
| 35% | Mix of α and β Ti | Mix of α and |
| | | martensitic Ti and |
| | | with some β Ti and |
| | | β Ta |
| 40-45% | Mix of α and β Ti | Predominantly |
| | | Martensitic with |
| | | some β Ta |
| 50-60% | Mix of α and β Ti | Predominantly β Ti |
| | | with some martensite. |

As shown in FIG. 3 and reflected in Table 2, the intensity vs. 2θ plots showed gradual but generally systemic shifts in phases present as Ta content varied from 20 to 60 weight %. In addition, martensitic peaks associated with the low-temperature phase transformation from austenite to martensite were identified, particularly in the TiTaO samples containing from 35 to 55 weight % tantalum.

The samples were also examined by energy-dispersive x-ray analysis (EDX) to determine their overall compositional variation from the targeted chemistry. The results of this analysis are reproduced in Table 3 below. As shown, the average EDX results were about 4% below the targeted compositions, which may be the result of the small size of the analyzed samples, or the production process.

TABLE 3
EDX Analysis of TiTaO Alloys Containing 0.2 wt % Oxygen

| | Target Ta | Area average | Ta | Ta |
|---|---|---|---|---|
| Sample | weight % | (weight %) | (% max) | (% min) |
| 1 | 20 | 16 | 17 | 15 |
| 2 | 25 | 24 | 20 | 22 |
| 3 | 30 | 25 | 30 | 26 |
| 4 | 35 | 29 | 35 | 29 |
| 5 | 40 | 37 | 57 | 36 |
| 6 | 45 | 41 | 47 | 39 |
| 7 | 50 | 45 | 48 | 41 |
| 8 | 55 | 52 | 86 | 45 |
| 9 | 60 | 56 | 57 | 48 |

EDX spatial maps of the produced alloys were also collected. These maps demonstrated that the homogeneity of the samples decreased as tantalum content increased.

The samples were also analyzed with optical microscopy to determine the morphology of the observed phases. Sample 2 (25 weight % Ta) contained a mixture of lamellar a phase with white specks of high tantalum β phase, and also showed a network of prior β grain boundaries. Sample 4 (25 weight % Ta) included α, β, and martensite phases, and looked similar to sample 2. However, the lamellar structure in sample 4 was more needle-like and thus, more consistent with a martensite structure. In addition, sample 4 showed evidence of lighter-etching regions associated with tantalum rich areas. Samples 7 and 9 (50 and 60 weight % Ta, respectively) contained β and martensite phases. The microstructure of these samples contained Ta rich beta phase islands surrounded by a needle-like martensitic “halo” in an otherwise all-β structure.

In sum, the XRD results and optical microscopy analysis showed clear evidence of the martensitic transformation associated with superelasticity and shape memory.

Mechanical and Elastic Properties of the Produced Samples

Miniature (3 mm by 0.7 mm) dog bone samples were used to obtain tensile data from the alloy samples described above. The Ultimate Tensile Strength (UTS), Yield Strength (YS), and elongation of these samples were measured. The results of these measurements were correlated against the bulk samples measured in accordance with ASTM Standard E-8, and were found to be accurate. The measured properties of samples 1-9 were compared to those of pure titanium and tantalum. The results of these analyses are reported in Table 4 below.

TABLE 4
Tensile Properties of TiTaO alloys containing
0.2 weight % Oxygen vs. Pure Ti and Pure Ta

| Sample | Weight % Ta | UTS (MPa) | YS (MPa) | Elongation (%) |
|---|---|---|---|---|
| 1 | 20 | — | 615 | — |
| 2 | 25 | 936 | 824 | 14 |
| 3 | 30 | 974 | 848 | 12 |
| 4 | 35 | 1043 | 854 | 8 |
| 5 | 40 | 1069 | 740 | 13 |
| 6 | 45 | 941 | 687 | 12 |
| 7 | 50 | 880 | 756 | 6 |
| 8 | 55 | 802 | 705 | 7 |
| 9 | 60 | 803 | 738 | 13 |
| Pure Ti | 0 | 980 | 910 | 14 |
| Pure Ta | 100 | 205 | 165 | 24 |

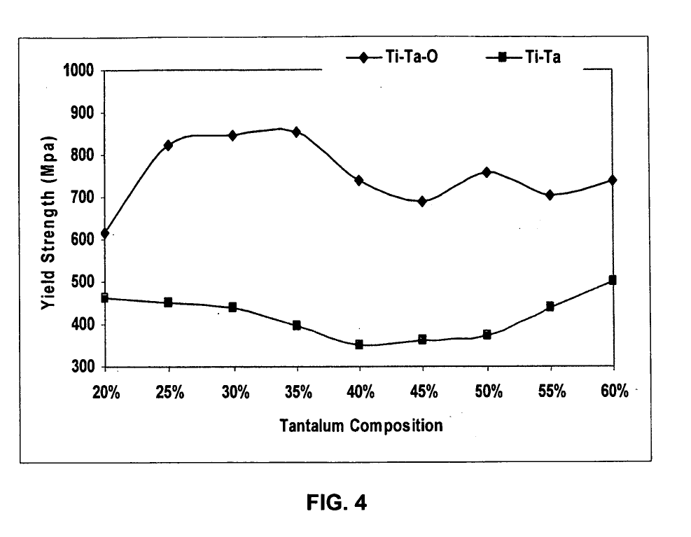

The Yield Strength (YS) of samples 1-9 was also compared to the reported Yield Strength of comparable binary TiTa alloys that contain less than 0.1 weight % oxygen. The results of this analysis are provided in Table 5 below and are represented graphically in FIG. 4. As shown, the Yield Strength of the TiTaO alloys according to the present disclosure was generally higher than that of corresponding binary TiTa alloys that contain less than 0.1 weight % oxygen.

TABLE 5
Yield Strength of TiTaO alloys vs. Binary TiTa alloys

| Sample | | YS | Comparative | | % difference in |
|---|---|---|---|---|---|
| # | % Ta | (MPA) | Sample # | YS (MPA)* | YS |
| 1 | 20% | 615 | 1 | 460 | 33.6 |
| 2 | 25% | 824 | 2 | 450 | 83.1 |
| 3 | 30% | 848 | 3 | 440 | 92.7 |
| 4 | 35% | 854 | 4 | 395 | 116.2 |
| 5 | 40% | 740 | 5 | 350 | 111.4 |
| 6 | 45% | 687 | 6 | 363 | 89.2 |
| 7 | 50% | 756 | 7 | 375 | 101.6 |
| 8 | 55% | 705 | 8 | 438 | 60.9 |
| 9 | 60% | 738 | 9 | 500 | 47.6 |

*reported in Zhou et al., “Effects of Ta Content on Young's Modulus and Tensile Properties of Binary Ti—Ta Alloys for Biomedical Applications,” Materials Science and Engineering A, Vol. 371, pp. 283-290 (2004).

The Young's Modulus, Shear Modulus, and Poisson's ratio of samples 1-9 was determined by sonic resonance testing using the impulse excitation technique described in ASTM Standards E1876 and C1259. The measured properties are reported in Table 6 below.

TABLE 6
The dynamic elastic properties of TiTaO alloys

| | | | Young's | Shear | |
|---|---|---|---|---|---|
| Sample | | Density | Modulus | Modulus | Poisson's |
| # | % Ta | (g/cm³) | (GPa) | (GPa) | ratio |
| 1 | 20% | 5.22 | 96.12 | 35.62 | 0.349 |
| 2 | 25% | 5.56 | 88.05 | 32.38 | 0.361 |
| 3 | 30% | 5.76 | 84.95 | 31.14 | 0.364 |
| 4 | 35% | 6.01 | 80.96 | 29.56 | 0.370 |
| 5 | 40% | 6.34 | 83.51 | 30.45 | 0.372 |
| 6 | 45% | 6.71 | 89.36 | 32.66 | 0.368 |
| 7 | 50% | 7.09 | 92.60 | 33.90 | 0.367 |
| 8 | 55% | 7.52 | 87.02 | 31.69 | 0.374 |
| 9 | 60% | 8.00 | 79.30 | 28.59 | 0.385 |

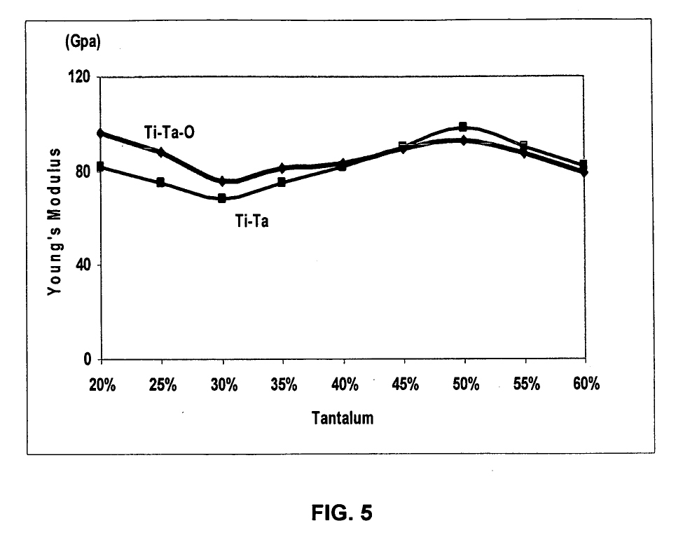

The measured Young's Modulus of samples 1-9 was compared to the Young's Modulus of comparable binary TiTa alloys. The results of this comparison are represented graphically in FIG. 5, which plots Young's Modulus vs. weight % Tantalum for the TiTaO alloys of the present disclosure and comparable binary TiTa alloys. As shown, the increased oxygen content of the TiTaO alloys had a negligible impact on Young's Modulus. As a result, the TiTaO alloys of the present disclosure exhibited higher strength-to-modulus ratios than comparable binary TiTa alloys.

The measured UTS, elastic modulus, and strength-to-modulus ratio of sample 3 (30 weight % Ta) was compared to binary Ti-30Ta, as well as other common alloys for biomedical implants. The results are shown in Table 7 below.

TABLE 7
Comparison of Ti—30Ta-0.2 Oxygen to
Known Biomedical Implant Materials

| | UTS | Elastic Modulus | Strength-to-modulus |
|---|---|---|---|
| Alloy | (MPa) | (GPa) | ratio |
| CP* Ti | 345 | 103 | 3.35 |
| F75 (CoCrMo) | 655 | 220 | 2.98-3.14 |
| 316 stainless | 580-1034 | 193 | 3.00-5.36 |
| steel | | | |
| TI—6Al—4V | 965 | 114 | 8.77 |
| Ti—6Al—7Nb | 960 | 111 | 8.65 |
| Ti—13Nb—13Zr | 862 | 79 | 10.91 |
| Ti—30Ta | 587 | 69 | 8.51 |
| Sample 3 | 951 | 76.4 | 12.4 |
| (Ti—30Ta-0.2 | | | |
| Oxygen) | | | |

As shown, the sample 3 (Ti-30Ta-0.2 Oxygen alloy formed by powder metallurgy and subsequent heat treatment) exhibited a strength-to-modulus ratio higher than most titanium alloys, while maintaining a low elastic modulus. That is, sample 3 exhibited a unique combination of high strength and low modulus, making it particularly suitable for use in biomedical implant applications.

Finally, two additional Ti-30Ta-0.2 Oxygen samples, samples 10 and 11, were produced. Sample 10 was produced by powder metallurgy followed by extrusion, whereas sample 11 was produced by powder metallurgy followed by extrusion and subsequent heat treatment. The tensile properties of samples 3, 10, and 11 were compared to those of binary Ti-30Ta. As shown in Table 8 below, the Ti-30Ta-0.2 Oxygen alloys exhibited strength superior to Ti-30Ta in all conditions, with equivalent ductility. In addition, samples 3, 10, and 11 all exhibited a strength-to-modulus ratio greater than that of Ti-30Ta.

TABLE 8
Comparison of Ti—30Ta-0.2 Oxygen Samples to Ti—30Ta

| | | | | Reduction | Young's | Strength-to- |
|---|---|---|---|---|---|---|
| | UTS | YS | Elongation | in Area | Modulus | modulus |
| Alloy | (MPa) | (MPa) | (%) | (%) | (GPa) | ratio |
| Sample 3* | 951 | 801 | 13 | 42 | 76 | 12.5 |
| Sample 10** | 880 | 838 | 20 | 70 | 51 | 17.3 |
| Sample 11*** | 881 | 726 | 20 | 56 | 92 | 9.6 |
| Ti—30Ta | 587 | 440 | 20 | — | 69 | 8.5 |

*Ti—30Ta-0.2 Oxygen produced by powder metallurgy and subsequent heat treatment
**Ti—30Ta-0.2 Oxygen produced by powder metallurgy and subsequent extrusion
***Ti—30Ta-0.2 Oxygen produced by powder metallurgy, subsequent extrusion and heat treatment

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A titanium tantalum oxygen alloy, said alloy comprising tantalum in an amount ranging from 20 to 60% by weight, at least 0.10 weight % oxygen, the balance comprising titanium and incidental impurities.

2. The alloy of claim 1, wherein said oxygen is present in an amount ranging from 0.10 to 0.30% by weight.

3. The alloy of claim 1, wherein said tantalum in present in an amount ranging from 20 to 40% by weight.

4. The alloy of claim 1, wherein said tantalum in present in an amount ranging from 40 to 60% by weight

5. The alloy of claim 4, wherein said alloy exhibits shape memory properties.

6. The alloy of claim 5, wherein said shape memory properties are chosen from superelasticity and austenite to martensite transformation.

7. The alloy of claim 1, wherein said alloy has a Young's Modulus ranging from 45 to 100 MPa.

8. The alloy of claim 1, wherein said alloy has an Ultimate Tensile Strength of at least 800 MPa.

9. The alloy of claim 1, wherein said alloy has a Yield Strength of at least 700 MPa.

10. The alloy of claim 1, wherein said alloy has a ductility of at least 17%.

11. The alloy of claim 1, said alloy having been prepared by powder metallurgy.

12. The alloy of claim 1, wherein said alloy has been heat treated at a temperature ranging from 1150° C. to 1250° C.

13. The alloy of claim 1, wherein said alloy has been hot worked by extrusion, rolling or forging.

14. An article comprising a titanium tantalum oxygen alloy, said alloy comprising titanium in an amount ranging from 40 to 80% by weight, tantalum in an amount ranging from 20 to 60% by weight, and oxygen in an amount ranging from 0.10 to 0.30% by weight.

15. The article of claim 14, wherein said article comprises a biomedical component chosen from a bone, spine, joint, dental, cardiovascular, and intravascular implant.

16. The article of claim 15, wherein said biomedical component comprises a rod, plate, disc, wire, fastener, cable, casting, valve, ring, stent, or basket.

17. The article of claim 16, wherein said fastener comprises a screw, nail, or anchor.

18. The article of claim 14, wherein said article is a component for the aerospace, automotive, nuclear, power generation, jewelry, communications, or chemical processing industry.

19. The article of claim 14, wherein said component is chosen from springs, torsions bars, fasteners, sheets, rings, wires, plates, rods, discs, and tubing.

20. A method of making a titanium tantalum oxygen alloy, said method comprising:
blending two or more powders, at least one powder comprising titanium and at least one powder comprising tantalum, wherein said powders are blended in a ratio to form an alloy comprising 20 to 60% by weight of tantalum,
compacting the blended powder;
heat treating the compacted and blended powder at a time and temperature sufficient to form a consolidated powder metal having at least 0.10 weight % oxygen.

21. The method of claim 20, wherein said oxygen is present in an amount ranging from 0.10 to 0.30% by weight.

22. The method of claim 20, wherein said tantalum in present in an amount ranging from 20 to 40% by weight.

23. The method of claim 20, wherein said tantalum in present in an amount ranging from 40 to 60% by weight.
